(19) United States
(12) Patent Application Publication
(10) Pub. No.: US 2011/0236356 A1
(43) Pub. Date: Sep. 29, 2011

(54) METHODS OF ISOLATING AND USING STEM CELLS

(75) Inventors: C.-Y. Charles Huang, Coral Gables, FL (US); Franklin Garcia-Godoy, Fort Lauderdale, TN (US); Herman S. Cheung, Miami, FL (US)

(73) Assignees: Nova Southeastern University, Fort Lauderdale, FL (US); University of Miami, Miami, FL (US)

(21) Appl. No.: 13/133,007

(22) PCT Filed: Dec. 14, 2009
(86) PCT No.: PCT/US09/67912

§ 371 Date: Jun. 6, 2011

Related U.S. Application Data
(60) Provisional application No. 61/122,231, filed on Dec. 12, 2008.
(60) Provisional application No. 61/237,083, filed on Aug. 26, 2009.

(51) Int. Cl. A61K 35/12; C12N 5/071; A61P 25/28; A61P 25/16; A61P 9/00; A61P 3/10; A61P 19/02

(52) U.S. Cl. 424/93.7; 435/325; 435/377

(57) ABSTRACT

Methods of isolating and using stem cells from neural crest, e.g. periodontal ligament, isolated stem cell and therapeutic cell cultures, and therapeutic applications for a variety of conditions. The cells and cell cultures are especially useful for autologous administration.

This application claims priority to U.S. provisional application No. 61/122,231, filed Dec. 12, 2009 and U.S. provisional application No. 61/237,083, filed Aug. 26, 209, each of which is incorporated by reference in its entirety.

The invention disclosed herein was developed in part using funds from the United States Department of Veterans Affairs. The U.S. Government has certain rights in the invention.

### BACKGROUND OF THE INVENTION

1. Field of the Invention

The invention relates to methods of isolating and using stem cells from neural crest, e.g. periodontal ligament; isolated stem cell and therapeutic cell cultures, and therapeutic applications for a variety of conditions, e.g. Parkinson's disease, Alzheimer's disease, a spinal cord injury, heart disease, diabetes, and osteoarthritis. The cells and cell cultures are especially useful for autologous administration.

2. Background Information

In spite of its origin from the ectoderm at the dorsal region of the neural tube, the neural crest (NC) contains pluripotent cells that contribute to the development of a wide variety of organs and tissues in the body after extensive migration. Depending on their final location, NC cells can give rise to neurons and glial cells of the peripheral nervous system, endocrine cells, connective tissue cells (e.g., ligament, cartilage, and bone), muscle cells, and pigment cells [1,2]. Based on regional characteristics and functions, the NC can be divided into four domains: cranial, trunk, vagal and sacral, and cardiac. Previous studies have demonstrated there may be an intrinsic disparity in the capability of cell differentiation among the NC regions, with the cranial NC region exhibiting a higher level of plasticity [1-3]. Stem cells derived from the NC may still reside in various types of NC derivatives and help tissue regeneration or repair throughout adulthood [4-12].

The periodontal ligament (PDL), which is derived from the cranial NC, is a soft connective tissue embedded between the tooth root and the alveolar bone socket. It contains heterogeneous cell populations including fibroblasts, endothelial cells, epithelial cell rests of Malassez, osteoblasts, and cementoblasts [13]. Due to the remarkable capability of PDL cells for renewal, it has been speculated that different cell types within the PDL may originate from progenitors already residing therein [10,13]. Recent studies have shown that the PDL contains multipotent stem cells that are able to differentiate into neural and mesenchymal lineages [10,14,15]. More recently, Ibi et al. were able to establish pluripotent cells lines from miniature swine PDL fibroblasts by gene transfection of a human telomerase reverse transcriptase [16]. However, pluripotency of human PDL cells has not yet been investigated.

Potential applications of pluripotent stem cells (e.g., embryonic stem cells or ESCs) include the development of cell-based regenerative therapies to treat diseases such as Parkinson's and Alzheimer's, spinal cord injury, heart disease, diabetes, and osteoarthritis. The transcription factors Oct4, Nanog, and Sox2 have been shown to be the key genes that lie at the core of the genetic circuitry involved in maintaining pluripotency of human ESCs [17-19]. Recent studies also demonstrated that pluripotent stem cells can be induced by introducing these key ESC genes into human dermal fibroblasts [20-23]. Therefore, the objective of our study was to identify subpopulations of stem cells from the adult PDL with the gene expressions of ESC and NC markers and investigate their pluripotency.

### BRIEF DESCRIPTION OF THE DRAWINGS

FIG. 1 (a) Colonies of PDL cells (Bar=200 μm). (b) Expressions of markers of ESC and NC in the PDL cells compared to those of hESCs. (c) Representative growth kinetics of PDL cells at passage 7 (n=4).

FIG. 2 (a) Immunofluorescence of PDL cells showing positive expression of ESC markers (Oct4, Sox2, Nanog, Klf4, SSEA3, SSEA4, and Tra-1-60) and Nestin. (b) Immunofluorescence of Oct4, Sox2, Nanog, SSEA3, SSEA4, and Tra-1-60 in ESCs for comparison (Bat-50 μm).

FIG. 3 (a) Gene expression of neurogenic markers (MAP2, GFAP, NF-M, and β-tubulin III) detected in the cells of ESC-M+ subpopulation after 2 weeks of culture under the condition favorable for neurogenic differentiation. (b) Immunofluorescence of β-tubulin III in the cells of ESC-M+ subpopulation after neurogenic differentiation (Bar=50 μm).

FIG. 4 (a) Gene expression of pancreatic islet markers (insulin, PDX1, somatostatin, GLUT2, glucagon). (b) Immunofluorescence of C-peptide in the cells of ESC-M+ subpopulation after 10 days of treatment for differentiation of insulin-producing cells (Bar=50 μm). Human pancreatic islet cells were used as positive controls. Comparison of (c) gene expression of cardiomyogenic markers MEF-2C, MYH7, HYL7, TNNT2, GATA-4, and NRx2.5/Csx and (d) immunofluorescence of sarcomeric α actinin (green) between the cells of ESC-M+ subpopulation with and without the treatment of 10 μM hydrogen peroxide (H₂O₂) for 8 days. In (d), the nuclei of the PDL cells were labeled with DAPI (blue).

FIG. 5 (a) Gene expression of osteogenic markers (ALP, RUNX2, OCN, OPN and ONN) detected in the cells of ESC-M+ subpopulation after 3-weeks of dexamethasone treatment. (b) Comparison on gene expression of Nestin, Oct4, Sox2, and Nanog between the PDL cells before and after 3-weeks of dexamethasone treatment. (c) Positive Alizarin red and von Kossa staining of calcium deposition on the culture of ESC-marker-positive PDL cells after 5 weeks of dexamethasone treatment. (d) Upregulation of chondrogenic gene expressions (aggrecan and collagen type II) in the ESC-maker-positive PDL cells after 2-weeks of TGF-β3 treatment.

### DESCRIPTION OF THE INVENTION

The present invention is directed to methods for isolating stem cells from neural crest tissues, such as periodontal ligament (PDL), cells and cell cultures so obtained, and methods of using those isolated stem cells as a therapeutic treatment. In some embodiments, the stem cells are isolated from a sample of PDL taken from a patient who will receive them as a therapeutic treatment. These patient-specific stem cell therapies can avoid the adverse and potentially fatal immunogenic reactions that can occur when foreign cells are introduced into a patient.

As one of skill in the art will appreciate, these methods have a wide array of uses. For example, these methods can be used to treat patients in need of treatment for Parkinson's disease, Alzheimer's disease, spinal cord injuries, heart disease, diabetes and osteoarthritis.

As described in more detail below, in some embodiments, the methods of isolating stem cells and for preparing therapeutic cells disclosed herein can begin by obtaining a sample of healthy PDL. The sample can be obtained from the patient who will receive the therapeutic cells, a donor, or other source of healthy PDL. Additional examples of suitable neural crest tissues are described in references 1 and 4-12 cited below.

The sample of PDL can then be digested, for example using an enzyme, placed into an appropriate cell culture medium, and passaged as needed to obtain a stable culture of PDL cells. One of skill in the art will appreciate that the cells may need to be cultured for a day, more than one day, one week, two weeks, three weeks, or more than four weeks to obtain a stable culture. Examples of suitable culturing methods and conditions are presented below, and are known to those of skill in the art.

The stable culture of PDL cells can then be screened for one or more cellular marker associated with embryonic stem cells. A cellular marker can be, for example, a molecule that allows for the detection and isolation of a particular cell type. For example, the protein Oct-4 can be used as a biomarker to identify embryonic stem cells.

In the present methods, the culture of PDL cells can be screened for an embryonic stem cell surface marker. By “embryonic stem cell surface marker” is meant a surface marker that is known to those of skill in the art to be specifically associated with embryonic stem cells. Suitable embryonic stem cell surface markers include, but are not limited to, SSEA-3, SSEA-4, TRA-1-60 and TRA-1-81. A variety of neural crest cell surface markers (34) should be suitable for screening.

The culture of PDL cells can also be screened for expression of a gene associated with stem cells. Suitable genes include, but are not limited to Oct4, Nanog, Sox2, Klf4, TERT, LIN-28, and alkaline phosphatase (ALP). Other examples will be known to those of skill in the art. (See, e.g. references 17-19 cited below).

The culture of PDL cells can also be screened for markers associated with the neural crest. For example, the cells can be screened for Nestin, Slug, Sox10, P75, and CD24. Other examples will be known to those of skill in the art (see, e.g. reference 34).

After the screening is complete, the cells expressing markers of interest can be isolated from the culture using the techniques described below and other techniques well known in the art. In some embodiments, these isolated cells expressing stem cell markers are pluripotent stem cells.

For therapeutic uses, in some embodiments, the pluripotent stem cells isolated from PDL are then differentiated into a desired cell type. As described below, the pluripotent stem cells can be differentiated into neurogenic, cardiomyogenic, chondrogenic, osteogenic, and insulin producing cells using the described methods. The pluripotent stem cells can be differentiated into cells of any of the three germ layers, i.e., ectoderm, mesoderm, or endoderm. As one of skill in the art will appreciate, the isolated stem cells can also be differentiated into other cells types using those methods known in the art.

The differentiated stem cells can then be tested to confirm their cellular profile is of therapeutic interest. For example, in seeking to treat a patient with diabetes mellitus the cells differentiated into insulin producing cells can be tested to confirm they produce insulin and are a good immunogenic match for the patient.

Acceptable differentiated cells can be administered to a person or mammal in need thereof using delivery methods known to one of skill in the art. For example, the administration can be performed using the compositions and methods of administration described in Remington: The Science and Practice of Pharmacy, 21^{st} Ed., Lippincott Williams and Wilkins (2005).

The examples set forth hereinbelow illustrate possible embodiments of the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents. Any headings used herein are provided solely for organizational purposes and are not intended to impart any division or meaning to this document, unless specifically indicated.

All documents cited anywhere in this document, including websites, journal articles or abstracts, published or corresponding U.S. or foreign patent applications, issued or foreign patents, or any other documents, are each entirely incorporated by reference herein, including all data, tables, figures, and text presented in the cited document.

### Examples

### Materials and Methods

### Isolation of PDL Cells

Human PDLs harvested from impacted wisdom teeth were collected from 3 patients (age 19-22 years) at the Clinic of the Nova Southeastern University College of Dental Medicine with their informed consent, according to approved institutional review board protocols. The PDLs from different teeth of the same donor were pooled and finely chopped, and cells released by overnight digestion at 37° C. in high glucose Dulbecco's Modified Eagle Medium (DMEM; Invitrogen Corp., Carlsbad, Calif.) supplemented with 10% fetal bovine serum (FBS; Invitrogen Corp.), 1% antibiotic-antimycotic, 1 mg/ml collagenase (Worthington Biochemical Corp., Lakewood, N.J.) and 0.6 mg/ml protease (Sigma-Aldrich Corp., St. Louis, Mo.). Single cell suspensions were obtained by passing the resulting digestion through a 70-μm cell strainer (BD Biosciences, Bedford, Mass.). Cells were plated on collagen-coated 6-well culture plates (at 1000 cells per well) in high glucose DMEM supplemented with 10% FBS and 1% antibiotics, and incubated at 37° C. in 5% CO₂. After 5 days of culture, nonadherent cells were discarded by changing the culture medium. After two weeks of primary culture, the PDL cells of each well (referred to as a subpopulation) were passaged into a T-75 culture flask (Passage 1). The PDL cells of each subpopulation were screened at passage 1 by examining the expression of Oct4, Nanog, Sox2, and Klf4, with human ESCs (H9 line) as a positive control. After screening, the ESC-marker-positive (ESC-M+) PDL cell subpopulations were expanded and examined at passages 3-5 for their capability of differentiating into derivatives of three germ layers (ectoderm, endoderm, and mesoderm). In addition to Oct4, Nanog, Sox2, and Klf4, expression of telomerase reverse transcriptase (TERT) gene and NC markers (i.e., Nestin, Slug, Sox10, and p75) was also analyzed for the PDL cells at Passage 1. All monolayer cultures were maintained subconfluent to prevent cell differentiation.

### Neurogenic Differentiation (Ectoderm)

PDL cells were cultured in 1% agarose-coated plates (non-adherent conditions) for 4 days with a chemically defined medium (Glasgow's modified Eagle's medium (GMEM) (Invitrogen) supplemented with 10% FBS, 0.1 mM β-mercaptoehtanol (Invitrogen), 1 mM sodium pryruvate, 1% non-essential amino acids (Invitrogen), 2 mM glutamine (Invitrogen), 0.1 mg/ml penicillin-streptomycin (Invitrogen), and B27 supplement (Invitrogen) containing 1 μM retinoid acid (Sigma-Aldrich Corp.) [6]. After 4 days, the PDL cells were transferred to gelatin-coated plates (monolayer culture) and cultured in the same chemical defined medium for 1 week. After 7 days of monolayer culture, neurogenic gene expression [MAP2, glial fibrillary acidic protein (GFAP), neurofilament (NF-M), and β-tubulin III] was analyzed. Immunocytostaining of β-tubulin III was also performed.

### Differentiation of Insulin-Producing Cells

Suspension culture of PDL cells was performed in 1% agarose-coated plates (non-adherent condition) as described in the previous section. The PDL cells were treated with DMEM/F12 (Invitrogen) supplemented with 1% non-essential amino acids (Invitrogen), 2 mM glutamine, 1% ITS+ Premix (final concentration: 6.25 μg/ml insulin, 6.25 μg/ml transferrin, 6.25 ng/ml selenous acid, 1.25 mg/ml bovine serum albumin and 5.35 μg/ml linoleic acid) (BD Biosciences), 450 μM monothioglycerol (Sigma-Aldrich Corp.), 1 mM sodium butyrate (Sigma-Aldrich Corp.), 10 mM nicotinamide (Sigma-Aldrich Corp.), and 5 mg/ml albumin fraction V (Sigma-Aldrich Corp.). After 10 days of suspension culture, expression of insulin, glucagon, pancreatic duodenum homeobox-1 (PDX-1), glucose transporter 2 (GLUT2), and somatostatin was analyzed and compared to that of human pancreatic islets cells. Immunocytostaining of C-peptide (a byproduct of insulin, to rule out uptake of exogenous insulin) was also carried out.

### Cardiomyogenic Differentiation (Mesoderm)

PDL cells were plated at a density of 2,500 cells/cm² in 6-well plates and cultured in low-glucose DMEM, 10% FBS, and 1% antibiotic-antimycotic. After initial overnight culture, the culture medium was supplemented with or without 10 μM hydrogen peroxide (Sigma-Aldrich Corp.) for the treated and control groups, respectively, and changed every other day to maintain the same levels of hydrogen peroxide. After 8 days of culture, Real Time RT-PCR analysis was performed to examine the expression of cardiomyogenic genes [myosin heavy (MYH7), light chains (MYL7), troponin T type 2 (cardiac; TNNT2), myocyte enhancer factor 2C (MEF-2C), GATA binding protein 4 (GATA-4), and cardiac homeobox gene NRx2.5/Csx]. Cells were also immunocytochemically stained for sarcomeric a actinin.

### Osteogenic Differentiation (Mesoderm)

PDL cells were plated in 12-well culture plates at a density of 40,000 cells/well and cultured in a basic serum-free medium (DMEM containing 50 μg/ml ascorbic acid (Sigma-Aldrich Corp.), 10 mM β-glycerophosphate (Sigma-Aldrich Corp.) and 1% antibiotic-antimycotic) supplemented with 100 nM dexamethasone (Sigma-Aldrich Corp.). After 3 weeks of culture, gene expressions of osteogenic markers [i.e., alkaline phosphatase (ALP), RUNX2, osteocalcin (OCN), osteopontin (OPN), and osteonectin (ONN)] were examined. Alizarin red and von Kossa staining of calcium deposition was done after 5 weeks of culture.

### Chondrogenic Differentiation (Mesoderm)

The chondrogenic potential of PDL cells was examined by pellet cultures. Cell pellets were formed by centrifuging 3×10⁵ cells in a 15-ml polypropylene tube and assigned to either control or treated group. The control group was cultured in serum-free medium consisting of high-glucose DMEM, 1% ITS+ Premix (BD Biosciences), and 50 μg/ml ascorbic acid (Sigma-Aldrich Corp.). The treated group was cultured in the same serum-free medium supplemented with 10 ng/ml of recombinant human TGF-β3 (Peprotech Inc., Minneapolis, Minn.). All pellet cultures were conducted in a humidified incubator maintained at 37° C. in 5% CO₂ for 14 days. The culture medium was changed every 2 to 3 days. Expression of chondrogenic genes collagen type II and aggrecan was examined after 14 days of culture.

### Reverse Transcription—Polymerase Chain Reaction (RT-PCR)

Total RNA was extracted using the reagent Trizol (Invitrogen) according to manufacturer's instructions. cDNA synthesis and PCR were performed with iScript cDNA synthesis kit and iQ Supermix (Bio-Rad Laboratories, Inc., Hercules, Calif.), respectively, using a thermal cycler (iCycler, Bio-Rad Laboratories, Inc.). PCR products were examined by agarose gel electrophoresis and stained by ethidium bromide. Gene expression of β-actin was used as an internal control. The sequences of PCR primers are shown in Table 1. The gene expressions of Oct4 and Nanog in the PDL cells were also examined by the TaqMan Gene Expression Assays (Applied Biosystems Inc., Foster City, Calif.; OCT4: Hs00742896_s1 and Nanog: Hs02387400_g1) using the StepOne Plus real-time PCR system (Applied Biosystems Inc).

TABLE 1
The PCR primer sequences

| Gene | Sequence | Size | GenBank |
|---|---|---|---|
| Oct4 (sense) | 5′- CTCCTGAAGCAGAAGAGGATCAC -3′ | 401 bp | NM_203289 |
| (antisense) | 5′- CTTCTGGCGCCGGTTACAGAACCA -3′ | | |
| Sox2 (sense) | 5′- TGCAGTACAACTCCATGACCA -3′ | 278 bp | NM_003106 |
| (antisense) | 5′- GTGCTGGGACATGTGAAGTCT -3′ | | |
| Nanog (sense) | 5′- GTCTTCTGCTGAGATGC -3′ | 353 bp | NM_024865 |
| (antisense) | 5′- AGTTGTTTTTCTGCCACC -3′ | | |
| Klf4 (sense) | 5′-ACGATCGTGGCCCCGGAAAAGGAC-3′ | 397 bp | NM_004235 |
| (antisense) | 5′-TGATTGTAGTGCTTTCTGGCTGGGCTCC-3′ | | |
| TERT (sense) | 5′- CCTGCTCAAGCTGACTCGACACCGTG -3′ | 446 bp | NM_198253 |
| (antisense) | 5′- GGAAAAGCTGGCCCTGGGGTGGAGC-3′ | | |
| Nestin (sense) | 5′-GCCCTGACCACTCCAGTTTA-3′ | 200 bp | NM_006617 |
| (antisense) | 5′-GGAGTCCTGGATTTCCTTCC-3′ | | |
| Slug (sense) | 5′-TGCTACACAGCAGCCAGATTCC-3′ | 383 bp | NM_003068 |
| (antisense) | 5′-TTTCTGGGCTGGCCAAACAT-3′ | | |
| Sox10 (sense) | 5′-TCTTGTAGTGGGCCTGGATGG-3′ | 303 bp | NM_006941 |
| (antisense) | 5′-TGAACGCCTTCATGGTGTGG-3′ | | |
| P75 (sense) | 5′-CTGCAAGCAGAACAAGCAAG -3′ | 310 bp | NM_002507 |
| (antisense) | 5′-GGCCTCATGGGTAAAGGAGT-3′ | | |
| MAP2 (sense) | 5′- CAGCAAAGGGATACTTTCAC -3′ | 496 bp | NM_002374 |
| (antisense) | 5′- ATGCTTTTTGTTGCTTCTTC-3′ | | |
| NF-M (sense) | 5′-GCTGCGTACAGAAAACTCCTG-3′ | 455 bp | NM_005382 |
| (antisense) | 5′-TCTTCGGCTTGGTCTGACTTA-3′ | | |
| GFAP (sense) | 5′-TCATCGCTCAGGAGGTCCTT-3′ | 382 bp | NM_002055 |
| (antisense) | 5′- CTGTTGCCAGAGATGGAGGTT-3′ | | |
| β-tubulin III (sense) | 5′-AGTGATGAGCATGGCATCGA-3′ | 317 bp | NM_006086 |
| (antisense) | 5′-AGGCAGTCGCAGTTTTCACA-3′ | | |
| Insulin (sense) | 5′- AGCCTTTGTGAACCAACACC -3′ | 245 bp | NM_000207 |
| (antisense) | 5′- GCTGGTAGAGGGAGCAGATG -3′ | | |
| PDX1 (sense) | 5′-ACCAAAGCTCACGCGTGGAAA-3′ | 200 bp | NM_002091 |
| (antisense) | 5′-GATGTGTCTCTCGGTCAAGTT-3′ | | |
| Somatostatin (sense) | 5′- GATGCTGTCCTGCCGCCTCC -3′ | 292 bp | NM_001048 |
| (antisense) | 5′- TGCCATAGCCGGGTTTGAG -3′ | | |
| GLUT2 (sense) | 5′- AGGACTTCTGTGGACCTTA TG-3′ | 231 bp | NM_000340 |
| (antisense) | 5′- GTTCATGTCAAAAAGCAGGG-3′ | | |
| Glucagon (sense) | 5′-AGGCAGACCCACTCAGTGA-3′ | 308 bp | NM_002054 |
| (antisense) | 5′-AACAATGGCGACCTCTTCTG-3′ | | |
| MYH7 (sense) | 5′- CTGGAG GCCGAGCAGAAGCGCAACG -3′ | 258 bp | NM_000257 |
| (antisense) | 5′- GTCCGCCCGCTCCTCTGCCTCATCC -3′ | | |
| TNNT2 (sense) | 5′- ATGAGCGGGAGAAGGAGCGGCAGAAC-3′ | 232 bp | NM_000364 |
| (antisense) | 5′- TCAATGGCCAGCACCTTCCTCCTCTC-3′ | | |
| MYL7 (sense) | 5′- GGGCCCCATCAACTTCACCGTCTTCC -3′ | 235 bp | NM_021223 |
| (antisense) | 5′- TGTAGTCGATGTTCCCCGCCAGGTCC -3′ | | |
| MEF-2C(sense) | 5′- GACTTTCTGAAGGATGGGCAA -3′ | 233 bp | NM_002397 |
| (antisense) | 5′- CAAGTGCTAAGCTTATCTCAGCA -3′ | | |
| GATA-4 (sense) | 5′-TCAAATTGGGATTTTCCGGA-3′ | 346 bp | NM_002052 |
| (antisense) | 5′-GCACGTAGACTGGCGAGGA-3′ | | |
| Nkx2.5/Csx (sense) | 5′-AGCCCTGGCTACAGCTGCA-3′ | 262 bp | NM_004387 |
| (antisense) | 5′-TGGGAGCCCCTTCTCCCCA-3′ | | |
| RUNX2 (sense) | 5′-TTCATCCCTCACTGAGAG-3′ | 354 bp | NM_004348 |
| (antisense) | 5′-TCAGCGTCAACACCATCA-3′ | | |
| ALP (sense) | 5′- GTTCAGCTCGTACTGCATGTC -3′ | 286 bp | NM_000478 |
| (antisense) | 5′- ATCGCCTACCAGCTCATGCAT -3′ | | |
| OPN (sense) | 5′-TGAAACGAGTCAGCTGGATG-3′ | 162 bp | BC022844 |
| (antisense) | 5′-TGAAATTCATGGCTGTGGAA-3′ | | |
| OCN (sense) | 5′- GGCAGCGAGGTAGTGAAGAG-3′ | 230 bp | NM_199173 |
| (antisense) | 5′- CTGGAGAGGAGCAGAACTGG-3′ | | |
| ONN (sense) | 5′-GTGCAGAGGAAACCGAAGAG-3′ | 172 bp | BC008011 |
| (antisense) | 5′-TCATTGCTGCACACCTTCTC-3′ | | |
| Collagen II (sense) | 5′-GAACCACTCTCACCCTTCACA-3′ | 285 bp | NM_001844 |
| (antisense) | 5′- GCCTCAAGGATTTCAAGGCAA-3′ | | |
| Aggrecan (sense) | 5′-TGAGGAGGGCTGGAACAAGTACC-3′ | 350 bp | NM_001135 |
| (antisense) | 5′-GGAGGTGGTAATTGCAGGGAACA-3′ | | |
| β-actin (sense) | 5′-CATGTACGTTGCTATCCAGGC-3′ | 250 bp | NM_001101 |
| (antisense) | 5′-CTCCTTAATGTCACGCACGAT-3′ | | |

### Immunocytochemistry

Cells were fixed in either 4% paraformaldehyde in phosphate buffered saline (PBS) at room temperature or ice-cold methanol at −20° C. Following two washes with PBS, the cells were blocked for 1 hour in PBS containing 1% bovine serum albumin (BSA) (Sigma-Aldrich Corp.) and 0.1% Triton X-100 (Sigma-Aldrich Corp.) for 45 minutes and then incubated in the primary antibody for 2 hours at room temperature. Following 3 washes with PBS, the cells were incubated in secondary antibody for 1 hour and nuclei were counterstained with DAPI (Invitrogen) for 10 min. After a final wash, the cells were imaged using an Olympus inverted fluorescent microscope. Primary antibodies used were SSEA-4 (1:100, Abcam Inc, Cambridge, Mass.), SSEA-3 (1:100, Abcam Inc), TRA-1-60 (1:200, Abcam Inc), Oct4 (1:200, Santa Cruz Biotechnology, Santa Cruz, Calif.), Sox2 (1:100, Santa Cruz Biotechnology, Inc), Nanog (1:50, Santa Cruz Biotechnology), Klf4 (1:100, Santa Cruz Biotechnology, Inc), βIII-tubulin (1:100, Sigma-Aldrich Corp., C-Peptide (1:100, Santa Cruz Biotechnology, Inc), and Sarcomeric α actinin (1:100, Abcam Inc). FITC-conjugated secondary antibodies included rabbit anti-mouse IgG+IgM+IgA (Abcam Inc), rabbit anti-rat IgG+IgM+IgA (Abcam Inc), goat anti-mouse IgM (Santa Cruz Biotechnology, Inc), goat anti-mouse IgG (Santa Cruz Biotechnology, Inc), and donkey anti-goat IgG (Santa Cruz Biotechnology, Inc).

### Proliferation Assay

Proliferation of PDL cells was monitored in four replicates over 9 days of culture using a TACS MTT cell proliferation and viability assay (R&D Systems, Inc., Minneapolis, Minn.). The absorbance of each well was determined spectrophotometrically at 600 nm using a plate reader (Dynex Technologies, Chantilly, Va.). The number of cells in each well was calculated based on a standard curve generated over a cell density range from 2.5×10³ to 6.5×10⁵ cells per well.

### Results

### Isolation of PDL Cells

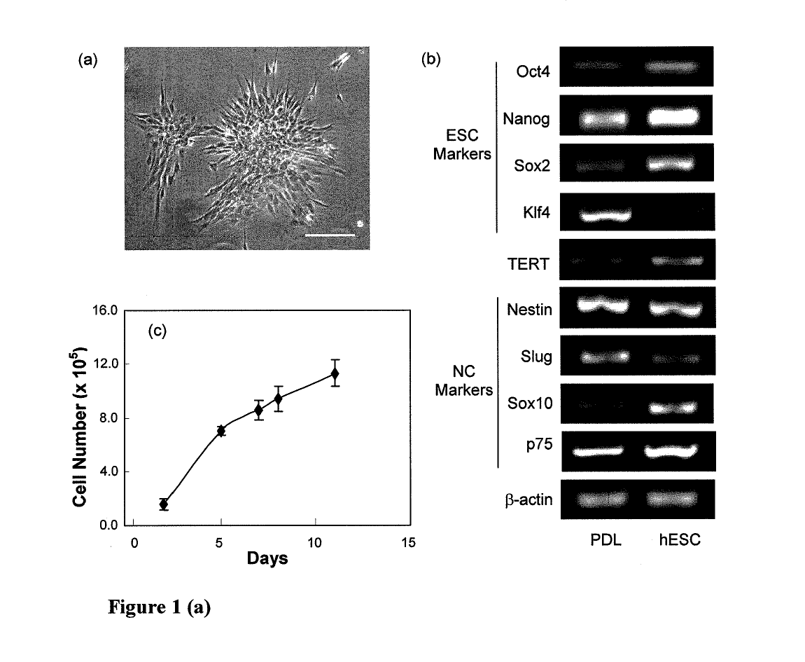

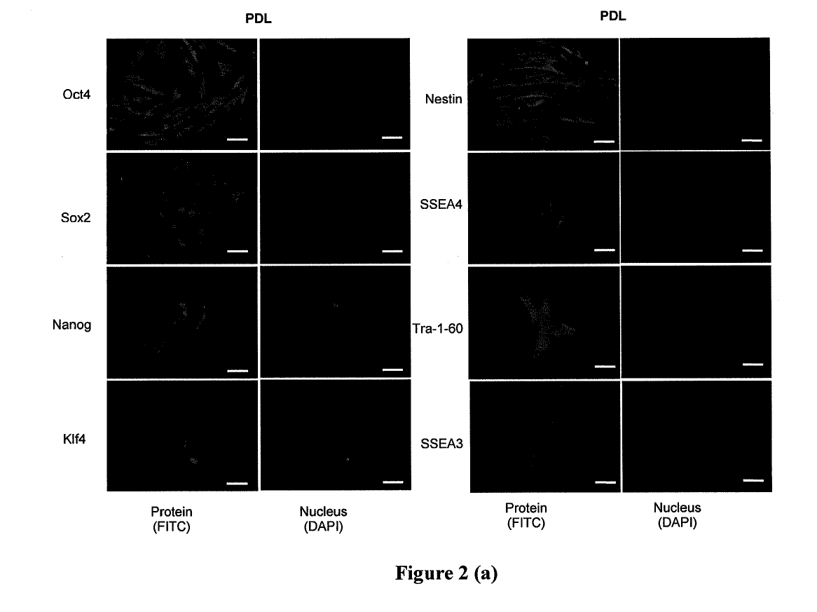

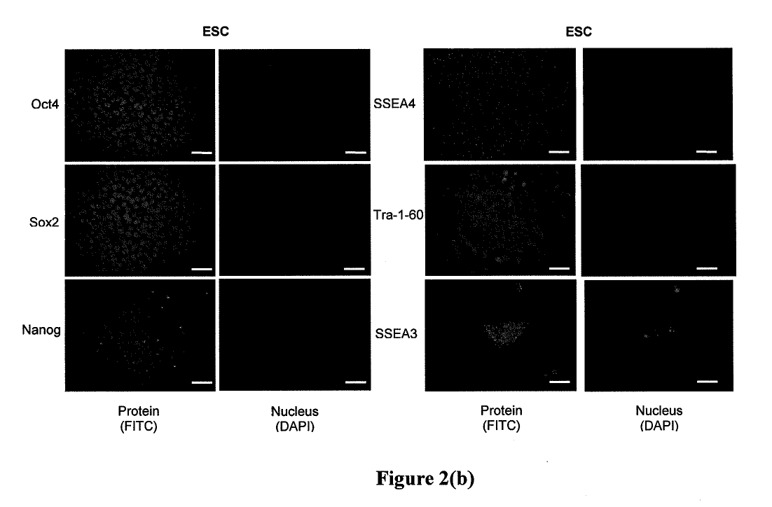

About 1 to 4 single-cell-derived colonies (≧50 cells) (FIG. 1a) were generated from 1000 cells that were initially seeded in one well of 6-well plate. Either one or multiple colonies formed in a well often yielded a continuous growing culture (a subpopulation). After screening 60 PDL subpopulations at the first passage, 56% of PDL subpopulations from 3 individuals were found to express all four key ESC genes: Oct4, Nanog, Sox2, and Klf4 (FIG. 1b). The gene expressions of Oct4 and Nanog in the PDL cells were further confirmed by the TaqMan Gene Expression Assays (Applied Biosystems Inc.; OCT4: Hs00742896_s1 and Nanog: Hs02387400_g1). Expression of TERT gene was also detected in the ESC-M+ cell subpopulation (FIG. 1b). However, with the exception of Klf4, the expression level of these genes was lower than in human ESCs. In addition, the ESC-M+ cell subpopulation expressed a subset of NC markers (i.e., Nestin, Slug, Sox10, and p75) (FIG. 1b) and showed a high proliferation rate, with a doubling time of 26.1 hours (FIG. 1c). Immunofluorescence (IF) analyses not only confirmed the expressions of Oct4, Nanog, Sox2, Klf4, and Nestin (FIG. 2a), but also showed weak positive expression of ESC-specific surface markers (i.e., SSEA-3, SSEA-4, TRA-1-60) in the ESC-M+ cell subpopulation (FIG. 2a). Secondary antibody-only controls showed no signal (data not shown). Immunofluorescence staining of Oct4, Sox2, Nanog, SSEA3, SSEA4, and Tra-1-60 in ESCs was shown in FIG. 2b for comparison. Of note, the pluripotency-associated transcription factors appeared to localize not only in the nucleus, but also in the cytoplasm. ESC-M+ cell subpopulations derived from either one or multiple colonies exhibited the same capability of multilineage differentiation that was demonstrated in the following sections.

### Neurogenic Differentiation

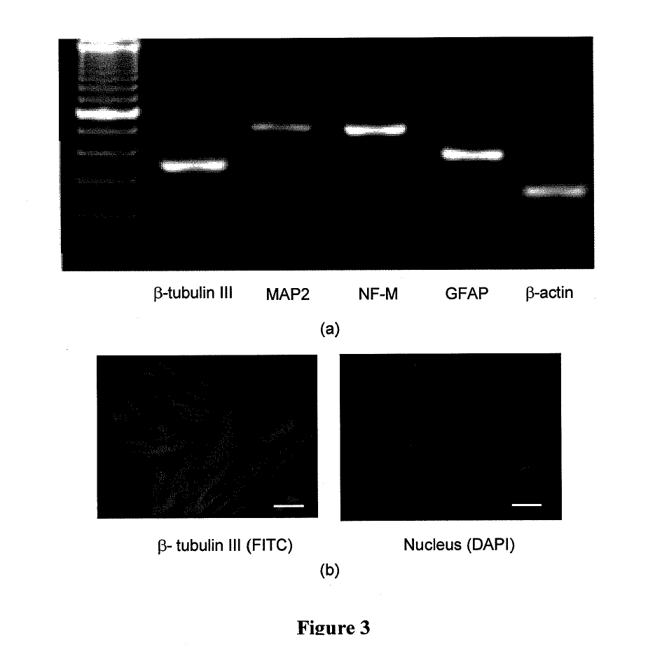

Following a neurogenic differentiation protocol reported previously in the study of Kerkis et al. [6] (see methods), aggregates of cells of ESC-M+ subpopulation were formed during 4 days of suspension culture. After transferring to gelatin-coated plates, cell aggregates attached to the plates within 24 hours and became proliferative during the following 7 days of monolayer culture. After 1 week in these conditions, the PDL cells expressed the neurogenic genes MAP2, GFAP, neurofilament (NF-M), and β-tubulin III (FIG. 3a) and showed strong IF signal of β-tubulin III (FIG. 3b).

### Differentiation of Insulin-Producing Cells

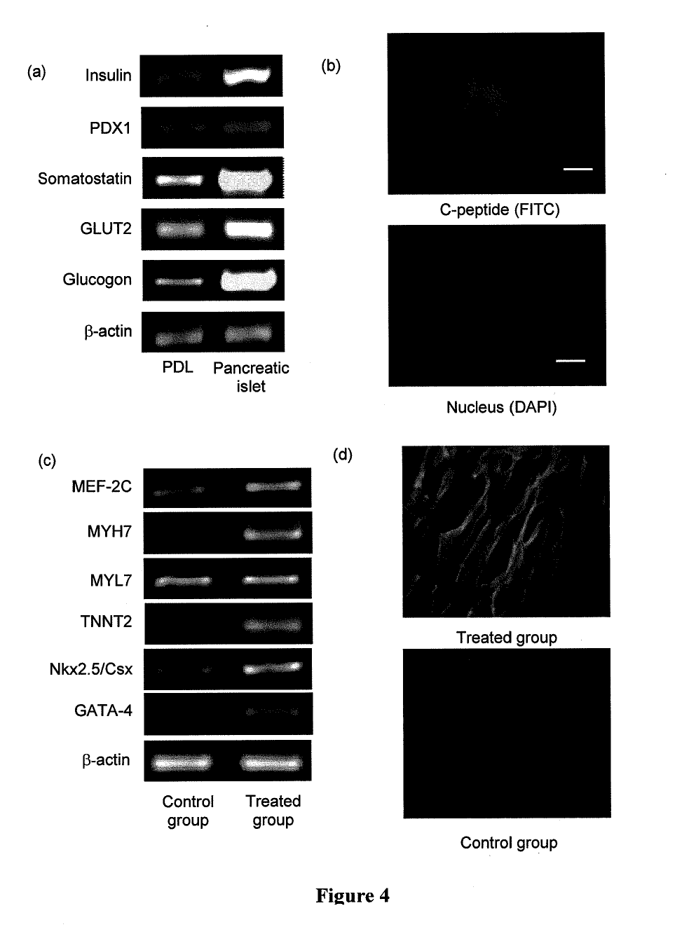

Monothioglycerol, sodium butyrate, and nicotinamide have been used to stimulate differentiation of ESCs into insulin-producing cells [24-27]. In this experiment, similar cell aggregates were formed within the first 24 hours of suspension culture. After 10 days of suspension culture in medium containing insulin, monothioglycerol, sodium butyrate, and nicotinamide, specific genes associated with pancreatic islet cells (i.e., insulin, PDX-1, GLUT2, and somatostatin) were detected (FIG. 4a) and positive IF signal of C-peptide (FIG. 4b) was also seen on the ESC-M+cell subpopulation. Secretion of C-peptide is an important criterion to claim insulin production from differentiated ESCs [24].

### Cardiomyogenic Differentiation

It has been shown that cardiomyogenesis of ESCs can be induced by low concentrations of reactive oxygen species such as hydrogen peroxide [28]. After 8 days of hydrogen peroxide treatment, cardiomyogenic gene expression (MYH7, MYL7, TNNT2, MEF-2C, GATA-4, and NRx2.5/Csx) of ESC-M+ cell subpopulation was detected (FIG. 4c). IF analysis showed that these cells were positive for sarcomeric α actinin (FIG. 4d).

### Osteogenic Differentiation

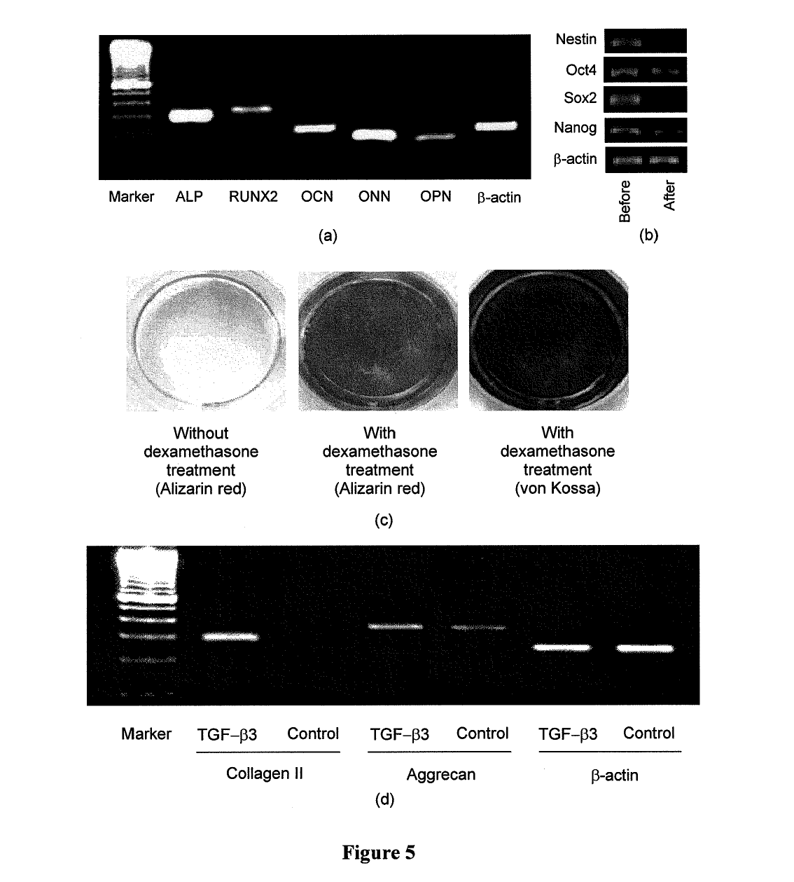

Dexamethasone is an osteogenic inducer for ESCs and bone marrow derived mesenchymal stem cells [29,30]. The osteogenic potential of ESC-M+ cell subpopulation was confirmed by positive gene expressions of osteogenic markers (i.e., ALP, RUNX2, OCN, OPN, and ONN) after 3-weeks of dexamethasone treatment (FIG. 5a). Gene expressions of ESC (Oct4, Sox2, and Nanog) and NC (Nestin) markers were downregulated in differentiated PDL cells (FIG. 5b). Strong Alizarin red and von Kossa staining of calcium deposition were seen on culture of ESC-M+ cell subpopulation after 5 weeks of dexamethasone treatment (FIG. 5c).

### Chondrogenic Differentiation

Transforming growth factor (TGF)-β is commonly used to induce chondrogenic differentiation of ESCs and bone marrow derived mesenchymal stem cells [30,31]. Chondrogenic differentiation of ESC-M+ cell subpopulation was induced by TGF-β3, which resulted in the upregulation of gene expression of collagen type II and aggrecan after 2 weeks of treatment (FIG. 5d).

### Discussion

The data presented above demonstrate that subpopulations of PDL cells expressed four major ESC markers (Oct4, Nanog, Sox2 and Klf4) and exhibited the potential to differentiate into neurogenic (ectoderm) as well as cardiomyogenic, osteogenic, and chondrogenic (mesoderm) cell lineages. Differentiation into insulin-producing derivatives is suggestive of pancreatic differentiation, indicating that PDL cells may be able to differentiate into the endodermal lineage. These findings suggest that the PDL may contain pluripotent stem cells. So far, human ESCs are the only pluripotent stem cells widely believed to have the potential to differentiate into derivatives of the three germ layers. However, leaving aside the ethical controversy on the derivation of ESCs, their clinical use will likely require immunosuppresion and involve the risk of spontaneous tumor formation. The PDL represents a reservoir of potentially pluripotent cells that could be isolated from each patient, thus providing an autologous source with none of the drawbacks of ESCs.

It has been shown that the pluripotency of human ESCs depends on the expression level of Oct4[32,33]. Although PDL cells express lower levels of Oct4, Nanog, and Sox2 than human ESCs, existence of pluripont cells in the PDL could be evidenced by their ability to differentiate in vitro along two of the three germ layers (ectoderm and mesoderm), and potentially endoderm. Since the culture conditions used in this study have not been optimized to maintain potential PDL pluripotent cells, they may gradually lose their pluripotency during cell isolation and initial monolayer culture. According to this hypothesis, ESC-like pluripotent stem cells may exist in the PDL, but their maintenance will likely require appropriate sorting and optimization of culture conditions. A recent study demonstrated that NC cells also expressed the ESC genes Oct4, Nanog, and Sox2[33]. The ESC-M+ cell subpopulation isolated in this study expressed several NC markers such as Nestin, Slug, Sox10, and p75. Since the PDL is derived from the cranial NC, the ESC-M+ cell subpopulation may be cranial NC-derived pluripotent stem cells as well. Previous animal studies showed that there were intrinsic differences in NC cell pluripotency [3,35-38]. The cells from the cranial NC exhibited a higher level of plasticity than the other NC cells. Since NC-derived stem cells may exist in different tissues after extensive migration during embryonic development [4-12], stem cells isolated from the derivatives of the cranial NC may be more capable of differentiating into various cell types. The hypothesis that the PDL cells herein described are pluripotent remnants of a more primitive cranial NC population certainly warrants additional studies. If proven true, their much easier accessibility through the PDL (which can be easily retrieved after routine extraction of wisdom teeth) would represent a breakthrough with major clinical implications.

ESC markers were found in both the nucleus and the cytoplasm of PDL cells. This localization pattern is different from that of ESCs, where the same markers were exclusively localized in the nucleus. However, this finding was supported by recent studies which also showed cytoplasmic localization of ESC markers Oct4[39,40] Sox2[41,42]. Nanog [43], and Klf4[44,45]. For instance, Sox2 was found to shuttle between the cytoplasm and nucleus during early embryogenesis [41]. A recent study demonstrated that human ESCs expressed two Oct4 isoforms which localized either in the nucleus (Oct4A) or the cytoplasm (Oct4B) [39].

The cranial NC is known to contribute to craniofacial development and can give rise to skeletal muscle, bone, and cartilage of the face [1]. The present study shows that the PDL-derived stem cells exhibit the same potential to differentiate into mesenchymal derivatives. This finding is also consistent with previous studies which demonstrated that stem cells isolated from different NC derivatives (such as hair follicle, periodontal ligament and dental pulp) can also differentiate along the chondrogenic and osteogenic lineages [5,9-11]. Furthermore, since the ESC-M+ cell subpopulations in this study expressed the markers of neural progenitors (i.e., Sox2 and Nestin), it is not surprising that they also had neurogenic potential. Again, this observation is also consistent with previous studies on stem cells derived from the periodontal ligament and dental pulp [6,14,15].

The data presented herein demonstrate that PDL-derived stem cells can differentiate into cardiomyocyte-like and insulin-producing cells. During cardiovascular development, cardiac NC cells migrating through the pharyngeal arches to the arterial pole of the heart contribute to the formation of the aortopulmonary septum and the cardiac neurons, differentiating into vascular smooth muscle cells of the aortic arch arteries [46-49]. A recent study showed that a subpopulation of multipotent stem cells with NC marker expression (i.e., Nestin, Musashi-1, and p75) isolated from the rat heart could differentiate into cardiomyocytes and nerve cells. These cells behaved like NC cells after transplantation into chick embryos, indicating that cardiac NC-derived stem cells may reside in the heart after migration [50]. The finding on cardiomyogenic differentiation of PDL cells in this study is supported by these previous studies, and suggests that NC-derived stem cell may exhibit cardiomyogenic potential.

Although the NC cells are involved in the development of the pancreas [51], whether or not the NC cells can give rise to its endocrine component remains controversial [52]. Recent reports that Nestin+ progenitor cells derived from the rat pancreatic islets may participate in the neogenesis of pancreatic endocrine cells through the Snail/Slug route [53,54] are in contradiction with basic developmental studies that seemingly exclude the endocrine lineage from a Nestin+ mesenchymal component [55]. Notwithstanding this, the ESC-M+ cell subpopulation isolated in this study expressed these two NC markers (Nestin and Slug) and had the potential to differentiate into insulin-producing cells, as previously reported from other Nestin+ populations [26,56]. These observations suggest that the NC derived stem cells may be a candidate cell source for the differentiation of insulin-producing cells. However, further studies are necessary both to ascertain the endodermal nature of these cells and to establish whether or not they are glucose-responsive.

Previous studies have demonstrated the pluripotency of adult human stem cells isolated from bone marrow, heart, and liver based on either the expression of specific surface makers [57], cell size [58], or survival under low oxygen culture conditions [59]. A different approach was used in this study, selecting subpopulations of pluripotent PDL stem cells by screening the expression of four genes (Oct4, Nanog, Sox2, and Klf4) that have been associated not only with the maintenance but also the induction of ESC phenotypes [20,21]. Surprisingly, this study found that more than 50% of isolated PDL cell subpopulations expressed ESC markers. Furthermore, unlike the specific culture conditions required for culture of adult pluripotent stem cells in previous studies [57-59], the general culture setting used in this study was shown to maintain multipotency of the PDL stem cells up to passage 7.

In summary, this study shows that subpopulations of PDL cells express ESC markers (Oct4, Sox2, Nanog, and Klf4) and exhibit a broad differentiation potential. The ESC-marker-positive PDL cells also express a subset of the NC makers Nestin, Slug, p75 and Sox10, indicating that they may originate from the NC. These observations are suggestive of a novel, easily retrievable reservoir of pluripotent cells that could potentially be used for autologous treatment.

### REFERENCES

1. S. F. Gilbert. Developmental Biology, eighth ed, Sinauer Associates, Inc., Sunderland, Mass., 2006.
2. N. M. Le Douarin, S. Creuzet, G. Couly, E. Dupin, Neural crest cell plasticity and its limits. Development 131 (2004) 4637-4650.
3. P. Y Lwigale, G. W. Conrad, M. Bronner-Fraser, Graded potential of neural crest to form cornea, sensory neurons and cartilage along the rostrocaudal axis. Development 131 (2004) 1979-1991.
4. K. J. Fernandes, I. A. McKenzie, P. Mill, K. M. Smith, M. Akhavan, F. Barnabé-Heider, J. Biernaskie, A. Junek, N. R. Kobayashi, J. G. Toma, D. R. Kaplan, P. A. Labosky, V. Rafuse, C. C. Hui, F. D. Miller, A dermal niche for multipotent adult skin-derived precursor cells. Nat. Cell Biol. 6 (2004) 1082-1093.
5. S. Gronthos, M. Mankani, J. Brahim, P. G. Robey, S. Shi, Postnatal human dental pulp stem cells (DPSCs) in vitro and in vivo. Proc. Natl. Acad. Sci. USA 97 (2000) 13625-13630.
6. I. Kerkis, A. Kerkis, D. Dozortsev, G. C. Stukart-Parsons, S. M. Gomes Massironi, L. V. Pereira, A. I. Caplan, H. F. Cerruti, Isolation and characterization of a population of immature dental pulp stem cells expressing OCT-4 and other embryonic stem cell markers. Cells Tissues Organs 184 (2006) 105-116.
7. G. M. Kruger, J. T. Mosher, S. Bixby, N. Joseph, T. Iwashita, S. J. Morrison, Neural crest stem cells persist in the adult gut but undergo changes in self-renewal, neuronal subtype potential, and factor responsiveness. Neuron 35 (2002) 657-669.
8. H. Y. Li, E. H. Say, X. F. Zhou. Isolation and characterization of neural crest progenitors from adult dorsal root ganglia. Stem Cells 25 (2007) 2053-2065.
9. M. Miura, S. Gronthos, M. Zhao, B. Lu, L. W. Fisher, P. G. Robey, S. Shi, SHED: stem cells from human exfoliated deciduous teeth. Proc. Natl. Acad. Sci. USA 100 (2003) 5807-5012.
10. B. M. Seo, M. Miura, S. Gronthos, P. M. Bartold, S. Batouli, J. Brahim, M. Young, P. G. Robey, C. Y. Wang, S. Shi, Investigation of multipotent postnatal stem cells from human periodontal ligament. Lancet 364 (2004) 149-155.
11. M. Sieber-Blum, M. Grim, Y. F. Hu, V. Szeder, Pluripotent neural crest stem cells in the adult hair follicle. Dev. Dyn. 231 (2004) 258-269.
12. C. E. Wong, C. Paratore, M. T. Dours-Zimmermann, A. Rochat, T. Pietri, U. Suter, D. R. Zimmermann, S. Dufour, J. P. Thiery, D. Meijer, F. Beermann, Y. Barrandon, L. Sommer, Neural crest-derived cells with stem cell features can be traced back to multiple lineages in the adult skin. J. Cell Biol. 75 (2006) 1005-1015.
13. P. Lekic, C. A. McCulloch, Periodontal ligament cell population: the central role of fibroblasts in creating a unique tissue. Anat. Rec. 245 (1996) 327-341.
14. W. Techawattanawisal, K. Nakahama, M. Komaki, M. Abe, Y. Takagi, I. Morita, Isolation of multipotent stem cells from adult rat periodontal ligament by neurosphere-forming culture system. Biochem. Biophys. Res. Commun. 357 (2007) 917-923.
15. D. Widera, W. D. Grimm, J. M. Moebius, I. Mikenberg, C. Piechaczek, G. Gassmann, N. A. Wolff, F. Thévenod, C. Kaltschmidt, B. Kaltschmidt, Highly efficient neural differentiation of human somatic stem cells, isolated by minimally invasive periodontal surgery. Stem Cells Dev. 16 (2007) 447-460.
16. M. Ibi, A. Ishisaki, M. Yamamoto, S. Wada, T. Kozakai, A. Nakashima, J. Iida, S. Takao, Y. Izumi, A. Yokoyama, M. Tamura. Establishment of cell lines that exhibit pluripotency from miniature swine periodontal ligaments. Arch Oral Biol. 52 (2007) 1002-8.
17. O. Adewumi, B. Aflatoonian, L. Ahrlund-Richter, M. Amit, P. W. Andrews, G. Beighton, P. A. Bello, N. Benvenisty, L. S. Berry, S. Bevan, B. Blum, J. Brooking, K. G. Chen, A. B. Choo, G. A. Churchill, M. Corbel, I. Damjanov, J. S. Draper, P. Dvorak, K. Emanuelsson, R. A. Fleck, A. Ford, K. Gertow, M. Gertsenstein, P. J. Gokhale, R. S. Hamilton, A. Hampl, L. E. Healy, O. Hovatta, J. Hyllner, M. P. Imreh, J. Itskovitz-Eldor, J. Jackson, J. L. Johnson, M. Jones, K. Kee, B. L. King, B. B. Knowles, M. Lako, F. Lebrin, B. S. Mallon, D. Manning, Y. Mayshar, R. D. McKay, A. E. Michalska, M. Mikkola, M. Mileikovsky, S. L. Minger, H. D. Moore, C. L. Mummery, A. Nagy, N. Nakatsuji, C. M. O'Brien, S. K. Oh, C. Olsson, T. Otonkoski, K. Y. Park, R. Passier, H. Patel, M. Patel, R. Pedersen, M. F. Pera, M. S. Piekarczyk, R. A. Pera, B. E. Reubinoff, A. J. Robins, J. Rossant, P. Rugg-Gunn, T. C. Schulz, H. Semb, E. S. Sherrer, H. Siemen, G. N. Stacey, M. Stojkovic, H. Suemori, J. Szatkiewicz, T. Turetsky, T. Tuuri, S. van den Brink, K. Vintersten, S. Vuoristo, D. Ward, T. A. Weaver, L. A. Young, W. Zhang, Characterization of human embryonic stem cell lines by the International Stem Cell Initiative. Nat. Biotechnol. 25 (2007) 803-816.
18. L. A. Boyer, T. I. Lee, M. F. Cole, S. E. Johnstone, S. S. Levine, J. P. Zucker, M. G. Guenther, R. M. Kumar, H. L. Murray, R. G. Jenner, D. K. Gifford, D. A. Melton, R. Jaenisch, R. A. Young, Core transcriptional regulatory circuitry in human embryonic stem cells. Cell 122 (2005) 947-956.
19. Y. H. Loh, Q. Wu, J. L. Chew, V. B. Vega, W. Zhang, X. Chen, G. Bourque, J. George, B. Leong, J. Liu, K. Y. Wong, K. W. Sung, C. W. Lee, X. D. Zhao, K. P. Chiu, L. Lipovich, V. A. Kuznetsov, P. Robson, L. W. Stanton, C. L. Wei, Y. Ruan, B. Lim, H. H. Ng, The Oct4 and Nanog transcription network regulates pluripotency in mouse embryonic stem cells. Nat. Genet. 38 (2006) 431-440.
20. I. H. Park, R. Zhao, J. A. West, A. Yabuuchi, H. Huo, T. A. Ince, P. H. Lerou, M. W. Lensch, G. Q. Daley, Reprogramming of human somatic cells to pluripotency with defined factors. Nature 451 (2008) 141-146.
21. K. Takahashi, K. Tanabe, M. Ohnuki, M. Narita, T. Ichisaka, K. Tomoda, S. Yamanaka, Induction of pluripotent stem cells from adult human fibroblasts by defined factors. Cell 131 (2007) 861-872.
22. J. Yu, M. A. Vodyanik, K. Smuga-Otto, J. Antosiewicz-Bourget, J. L. Frane, S. Tian, J. Nie, G. A. Jonsdottir, V. Ruotti, R. Stewart, I. I. Slukvin, J. A. Thomson, Induced pluripotent stem cell lines derived from human somatic cells. Science 318 (2007) 1917-1920.
23. M. Nakagawa, M. Koyanagi, K. Tanabe, K. Takahashi, T. Ichisaka, T. Aoi, K. Okita, Y. Mochiduki, N. Takizawa, S. Yamanaka, Generation of induced pluripotent stem cells without Myc from mouse and human fibroblasts. Nat. Biotechnol. 26 (2008) 101-106.
24. M. Hansson, A. Tonning, U. Frandsen, A. Petri, J. Rajagopal, M. C. Englund, R. S. Heller, J. Hakansson, J. Fleckner, H. N. Skold, D. Melton, H. Semb, P. Serup, Artifactual insulin release from differentiated embryonic stem cells. Diabetes 53 (2004) 2603-2609.
25. H. T. Ku, N. Zhang, A. Kubo, R. O'Connor, M. Mao, G. Keller, J. S. Bromberg, Committing embryonic stem cells to early endocrine pancreas in vitro. Stem Cells 22 (2004) 1205-1217.
26. N. Lumelsky, O. Blondel, P. Laeng, I. Velasco, R. Ravin, R. McKay, Differentiation of embryonic stem cells to insulin-secreting structures similar to pancreatic islets. Science 292 (2001) 1389-1394.
27. E. McKiernan, L. O'Driscoll, M. Kasper, N. Barron, F. O'Sullivan, M. Clynes, Directed differentiation of mouse embryonic stem cells into pancreatic-like or neuronal- and glial-like phenotypes. Tissue Eng. 13 (2007) 2419-2430.
28. H. Sauer, G. Rahimi, J. Hescheler, M. Wartenberg, Effects of electrical fields on cardiomyocyte differentiation of embryonic stem cells. J. Cell Biochem. 75 (1999) 710-723.
29. R. C. Bielby, A. R. Boccaccini, J. M. Polak, L. D. Buttery, In vitro differentiation and in vivo mineralization of osteogenic cells derived from human embryonic stem cells. Tissue Eng. 10 (2004) 1518-1525.
30. M. F. Pittenger, A. M. Mackay, S. C. Beck, R. K. Jaiswal, R. Douglas, J. D. Mosca, M. A. Moorman, D. W. Simonetti, S. Craig, D. R. Marshak, Multilineage potential of adult human mesenchymal stem cells. Science 284 (1999) 143-7.
31. N. S. Hwang, M. S. Kim, S. Sampattavanich, J. H. Baek, Z. Zhang, J. Elisseeff, Effects of three-dimensional culture and growth factors on the chondrogenic differentiation of murine embryonic stem cells. Stem Cells 24 (2006) 284-291.
32. V. Camara-Clayette, F. Le Pesteur, W. Vainchenker, F. Sainteny, Quantitative Oct4 overproduction in mouse embryonic stem cells results in prolonged mesoderm commitment during hematopoietic differentiation in vitro. Stem Cells 24 (2006) 1937-1945.
33. R. T. Rodriguez, J. M. Velkey, C. Lutzko, R. Seerke, D. B. Kohn, K. S. O'Shea, M. T. Firpo, Manipulation of OCT4 levels in human embryonic stem cells results in induction of differential cell types. Exp. Biol. Med. (Maywood) 232 (2007) 1368-1380.
34. S. Thomas, M. Thomas, P. Wincker, C. Babarit, P. Xu, M. C. Speer, A. Munnich, S. Lyonnet, M. Vekemans, H. C. Etchevers, Human neural crest cells display molecular and phenotypic hallmarks of stem cells. Hum. Mol. Genet. (2008) 17 (21):3411-3425.
35. N. M. Le Douarin, M. A, Teillet, Experimental analysis of the migration and differentiation of neuroblasts of the autonomic nervous system and of neurectodermal mesenchymal derivatives, using a biological cell marking technique. Dev. Biol. 41 (1974) 162-184.
36. C. S. Le Lievre, G. G. Schweizer, C. M. Ziller, N. M Le Douarin, Restrictions of developmental capabilities in neural crest cell derivatives as tested by in vivo transplantation experiments. Dev. Biol. 77 (1980) 362-378.
37. H. Nakamura, C. S. Ayer-Le Lievre, Mesectodermal capabilities of the trunk neural crest of birds. J. Embryol. Exp. Morphol. 70 (1982) 1-18.
38. D. M. Noden, The control of avian cephalic neural crest cytodifferentiation. I. Skeletal and connective tissues. Dev. Biol. 67 (1978) 296-312.
39. G. Cauffman, I. Liebaers, A. Van Steirteghem, H. Van de Velde, POU5F1 isoforms show different expression patterns in human embryonic stem cells and preimplantation embryos. Stem Cells 24 (2006) 2685-2691.
40. J. Lee, H. K. Kim, J. Y. Rho, Y. H. Han, J. Kim, The human OCT-4 isoforms differ in their ability to confer self renewal. J. Biol. Chem. 281 (2006) 33554-3365.
41. A. A. Avilion, S. K. Nicolis, L. H. Pevny, L. Perez, N. Vivian, R. Lovell-Badge, Multipotent cell lineages in early mouse development depend on SOX2 function. Genes Dev. 17 (2003) 126-140.
42. E. T. Park, J. R. Gum, S. Kakar, S. W. Kwon, G. Deng, Y. S. Kim, Aberrant expression of SOX2 upregulates MUC5AC gastric foveolar mucin in mucinous cancers of the colorectum and related lesions. Int. J. Cancer 122 (2008) 1253-1260.
43. U. I. Ezeh, P. J. Turek, R. A. Reijo, A. T. Clark, Human embryonic stem cell genes OCT4, NANOG, STELLAR, and GDF3 are expressed in both seminoma and breast carcinoma. Cancer 104 (2005) 2255-2265.
44. R. Behr, C. Deller, M. Godmann, T. Müller, M. Bergmann, R. Ivell, K. Steger, Kruppel-like factor 4 expression in normal and pathological human testes. Mol. Hum. Reprod. 13 (2007) 815-820.
45. A. Y. Pandya, L. I. Talley, A. R. Frost, T. J. Fitzgerald, V. Trivedi, M. Chakravarthy, D. C. Chhieng, W. E. Grizzle, J. A. Engler, H. Krontiras, K. I. Bland, A. F. LoBuglio, S. M. Lobo-Ruppert, J. M., Ruppert, Nuclear localization of KLF4 is associated with an aggressive phenotype in early-stage breast cancer. Clin. Cancer. Res. 10 (2004) 2709-2719.
46. S. J. Conway, D. J. Henderson, A. J. Copp, Pax3 is required for cardiac neural crest migration in the mouse: evidence from the splotch (Sp2H) mutant. Development 124 (1991) 505-514.
47. X. Jiang, D. H. Rowitch, P. Soriano, A. P. McMahon, H. M. Sucov, Fate of the mammalian cardiac neural crest. Development 127 (2000) 1607-1616.
48. M. L. Kirby, T. F. Gale, D. E. Stewart, Neural crest cells contribute to normal aorticopulmonary septation. Science 220 (1983) 1059-1061.
49. F. A. High, M. Zhang, A. Proweller, L. Tu, M. S. Parmacek, W. S. Pear, J. A. Epstein, An essential role for Notch in neural crest during cardiovascular development and smooth muscle differentiation. J. Clin. Invest. 117 (2007) 353-363.
50. Y. Tomita, K. Matsumura, Y. Wakamatsu, Y. Matsuzaki, I. Shibuya, H. Kawaguchi, M. Ieda, S. Kanakubo, T. Shimazaki, S. Ogawa, N. Osumi, H. Okano, K. Fukuda, Cardiac neural crest cells contribute to the dormant multipotent stem cell in the mammalian heart. J. Cell Biol. 170 (2005) 1135-1146.
51. N. Nekrep, J. Wang, T. Miyatsuka, M. S. German, Signals from the neural crest regulate beta-cell mass in the pancreas. Development 135 (2008) 2151-2160.
52. A. Andrew, B. Kramer, B. B. Rawdon, The origin of gut and pancreatic neuroendocrine (APUD) cells—the last word? J. Pathol. 186 (1998) 117-118.
53. J. M. Rukstalis, J. F. Habener, Snail2, a mediator of epithelial-mesenchymal transitions, expressed in progenitor cells of the developing endocrine pancreas. Gene. Expr. Patterns 7 (2007) 471-479.
54. H. Zulewski, E. J. Abraham, M. J. Gerlach, P. B. Daniel, W. Moritz, B. Müller, M. Vallejo, M. K. Thomas, J. F. Habener, Multipotential nestin-positive stem cells isolated from adult pancreatic islets differentiate ex vivo into pancreatic endocrine, exocrine, and hepatic phenotypes. Diabetes 50 (2001) 521-533.
55. L. Selander, H. Edlund, H., Nestin is expressed in mesenchymal and not epithelial cells of the developing mouse pancreas. Mech. Dev. 113 (2002) 189-192.
56. Y. Hori, X. Gu, X. Xie, S. K. Kim, Differentiation of insulin-producing cells from human neural progenitor cells. PLoS Med. 2 (2005) e103.
57. R. E. Schwartz, M. Reyes, L. Koodie, Y. Jiang, M. Blackstad, T. Lund, T. Lenvik, S. Johnson, W. S. Hu, C. M. Verfaillie, C. M., Multipotent adult progenitor cells from bone marrow differentiate into functional hepatocyte-like cells. J. Clin. Invest. 109 (2002) 1291-1302.
58. A. P. Beltrami, D. Cesselli, N. Bergamin, P. Marcon, S. Rigo, E. Puppato, F. D'Aurizio, R. Verardo, S. Piazza, A. Pignatelli, A. Poz, U. Baccarani, D. Damiani, R. Fanin, L. Mariuzzi, N. Finato, P. Masolini, S. Burelli, O. Belluzzi, C. Schneider, C. A. Beltrami, Multipotent cells can be generated in vitro from several adult human organs (heart, liver, and bone marrow). Blood 110 (2007) 3438-3446.
59. G. D'Ippolito, S. Diabira, G. A. Howard, P. Menei, B. A. Roos, P. C. Schiller, Marrow-isolated adult multilineage inducible (MIAMI) cells, a unique population of postnatal young and old human cells with extensive expansion and differentiation potential. J. Cell Sci. 117 (2004) 2971-2981.

We claim:

1. A method for isolating stem cells comprising: providing an initial cell culture of tissue derived from the cranial neural crest; screening the initial cell culture for those cells expressing a marker of interest associated with stem cells; and isolating the cells expressing the stem cell marker of interest to obtain an isolated stem cell culture

2. The method of claim 1, wherein the tissue derived from the cranial neural crest is periodontal ligament.

3. The method of claim 1, wherein the marker is an embryonic stem cell surface marker.

4. The method of claim 3, wherein the embryonic stem cell surface marker is selected from the group consisting of SSEA-3, SSEA-4, TRA-1-60 and TRA-1-81.

5. The method of claim 1, wherein the marker is the expression of a gene.

6. The method of claim 5, wherein the gene is selected from the group consisting of Oct4, Nanog, Sox2, Klf4, TERT, LIN-28 and alkaline phosphatase (ALP).

7. The method of claim 1, wherein cells expressing more than one marker of interest are isolated.

8. An isolated stem cell culture produced by the method of claim 1.

9. A method of treating a disease or disorder comprising administering an effective amount of the cell culture according to claim 8 to a subject in need of treatment.

10. The method of claim 9, wherein the initial cell culture is obtained from the subject.

11. The method of claim 9, wherein the disease or disorder is selected from the group consisting of Parkinson's disease, Alzheimer's disease, a spinal cord injury, heart disease, diabetes, and osteoarthritis.

12. A method of producing isolated therapeutic cells comprising:
a) screening a culture of periodontal ligament cells for those cells expressing a marker of interest associated with stem cells;
b) isolating the cells expressing the marker of interest; and
c) differentiating the isolated cells into therapeutic cells.

13. The method of claim 12, wherein the marker of interest is selected from the group consisting of SSEA-3, SSEA-4, TRA-1-60 and TRA-1-81.

14. The method of claim 12, wherein the therapeutic cells are selected from the group consisting of neurogenic, cardiomyogenic, chondrogenic, osteogenic, and insulin producing cells.

15. Isolated therapeutic cells produced by the method of claim 12.

16. A method of treating a disease or disorder, comprising administering an effective amount of the isolated therapeutic cells of claim 15 to a patient in need of treatment.

17. The method of claim 16, wherein the culture of periodontal ligament cells is generated from a sample of periodontal ligament from the patient.

18. The method of claim 17, wherein the disease or medical condition is selected from the group consisting of Parkinson's disease, Alzheimer's disease, a spinal cord injury, heart disease, diabetes, and osteoarthritis.
